(19) United States
(12) Patent Application Publication
(10) Pub. No.: US 2010/0196424 A1
(43) Pub. Date: Aug. 5, 2010

(54) STABILIZATION OF LONG CHAIN POLYUNSATURATED OILS

(76) Inventor: Dharma R. Kodali, Minneapolis, MN (US)

Correspondence Address: Gary C. Cohn, PLLC, 215 E. 96TH ST., #19L, New York, NY, 10128, US

(21) Appl. No.: 12/670,516

(22) PCT Filed: Jul. 25, 2008
(86) PCT No.: PCT/US08/71178

§ 371 Date: Jan. 25, 2010

Related U.S. Application Data
(60) Provisional application No. 60/952,670, filed on Jul. 30, 2007.

(51) Int. Cl. A61K 9/00; A61K 31/20; A61P 31/18

(52) U.S. Cl. 424/400; 514/560

(57) ABSTRACT

Compositions that include an oxidizable fatty acid-containing oil and wax capable of limiting oxidation of the oil are disclosed. Also disclosed are compositions that include an ingestible fatty acid-containing oil and an ingestible wax. Polyunsaturated fatty acid-containing oils may be stabilized by mixing the oil with a melted wax capable of limiting oxidation of the oil, and allowing the mixture to cool.

### CROSS-REFERENCE TO RELATED APPLICATION

This application claims priority to U.S. Provisional Patent Application Ser. No. 60/952,670, filed Jul. 30, 2007.

### BACKGROUND

Long chain polyunsaturated fatty acids include straight chain fatty acids of 18 to 24 carbon atoms containing multiple double bonds. Certain polyunsaturated fatty acids may be classified as omega-6 fatty acids or omega-3 fatty acids, based on whether the last double bond is six carbons or three carbons, respectively, from the terminal methyl group. An example of an omega-6 polyunsaturated fatty acid is arachidonic acid (C20:4). Omega-3 fatty acids are essential fatty acids required for human health. Long chain omega-3 polyunsaturated fatty acids such as eicosapentaenoic acid (EPA, C20:5) or docosahexaenoic acid (DHA, C22:6) can be derived from, for example, fish and algal sources. These fatty acids are important for growth, brain function, and visual acuity. Also, omega-3 fatty acid-containing oils can reduce the risk of a variety of ailments such as, for example, Alzheimer's disease, inflammation, and cardiovascular diseases.

Because they are polyunsaturated, omega-3 fatty acid-containing oils are susceptible to oxidation. Oxidation leads to the formation of hydroperoxides, loss of desired functionality, and loss of certain organoleptic characteristics such as, for example, taste, color, odor, and/or feel. Lipid oxidation can limit the desirability of using omega-3 fatty acid-containing oils in, for example, processed foods, drinks, and nutritional supplements. Oxidation of long chain polyunsaturated fatty acids can result in the development of undesirable sensory attributes due, for example, to the accumulation of hydroperoxides and their subsequent secondary products. In order to reduce oxidation of the polyunsaturated fatty acids, products that contain omega-3 fatty acids may be fortified with natural antioxidants (e.g., tocopherols, green tea extracts, rosemary extracts, etc.) and/or synthetic antioxidants such as, for example, butylated hydroxyanisole (BHA), butylated hydroxytoluene (BHT) and tert-butylhydroquinone (TBHQ).

The initial step in the process by which polyunsaturated fatty acids are oxidized is the formation of peroxide. The association between lipid peroxidation and diseases such as atherosclerosis, diabetes, Alzheimers, cancer and AIDS is well-known. Lipid peroxidation is initiated by a reactive oxygen species that reacts with the methylene adjacent to the double bond in the acyl chain to form hydroperoxide. The bis-allylic methylenes present in the polyunsaturated fatty acids are the most susceptible sites for oxidation. Thus, peroxide formation can provide a good measure of oxidation potential of a substrate.

### SUMMARY

The present invention provides a method of stabilizing polyunsaturated fatty acid-containing oils. Generally, the method includes mixing a polyunsaturated fatty acid-containing oil and melted wax; and allowing the mixture to cool.

The present invention also provides a composition that includes an oxidizable fatty acid-containing oil and a wax capable of limiting oxidation of the oil.

The present invention also provides a composition that includes an ingestible fatty acid-containing oil encapsulated in an ingestible wax.

Various other features and advantages of the present invention should become readily apparent with reference to the following detailed description, examples, and claims. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

This invention relates to compositions that include a polyunsaturated fatty acid-containing oil in which the composition is designed to limit oxidation of the fatty acid-containing oil. A fatty acid-containing oil can be incorporated into many types of foods, nutritional supplements, and cosmetics, for example. However, over time, the fatty acid-containing oil can be oxidized, which can cause a product containing the oil to develop an undesirable sensory (i.e., organoleptic) property such as, for example, an undesirable taste, odor, color and/or feel. The compositions of the invention can limit the oxidation of a fatty acid-containing oil and, when used in a product, can increase the length of time that the product remains usable before oxidation of the fatty acid-containing oil causes undesirable organoleptic properties to develop. Thus, the present invention provides a composition that generally includes an oxidizable polyunsaturated fatty acid-containing oil and a wax capable of reducing oxidation of the oil. In another aspect, the invention provides a composition that generally includes an ingestible polyunsaturated fatty acid-containing oil encapsulated in an ingestible wax.

In another aspect, the present invention also relates to methods of forming a composition that includes a fatty acid-containing oil so that oxidation of the fatty acid-containing oils is limited. Thus, the present invention provides a method of stabilizing a polyunsaturated fatty acid-containing oil. Generally, the method includes mixing a polyunsaturated fatty acid-containing oil and melted wax and allowing the mixture to cool.

The fatty acid-containing oil can be any polyunsaturated fatty acid-containing oil. In certain embodiments, the fatty acid-containing oil can be a long chain polyunsaturated fatty acid-containing oil. As used herein, “long chain polyunsaturated fatty acid-containing oil” refers to a mono- or polyol-ester of a fatty acid having a chain length of from 18 to 24 carbons and three or more double bonds. Oils having at least 10% of their fatty acid chains meeting these criteria are considered to be long chain polyunsaturated fatty acid-containing oils. Such oils may be naturally-occurring, derivatives of naturally-occurring oils, or synthetic.

In certain embodiments, polyunsaturated fatty acid-containing oil can include an omega-3 fatty acid-containing oil or an omega-6 fatty acid-containing oil. The health benefits of such oils are well documented and include, for example, hypolipidemic, anti-thrombotic, and anti-inflammatory properties. They are also essential for growth, brain function, and visual acuity. As their name suggests, omega-3 fatty acid-containing oils include at least one omega-3 fatty acid. Suitable omega-3 fatty acids include, for example, eicosapentaenoic acid, docosahexaenoic acid, or any combination thereof. Similarly, omega-6 fatty acid-containing oils include at least one omega-6 fatty acid. Suitable omega-6 fatty acids include, for example, arachidonic acid.

The polyunsaturated fatty acid-containing oil can be obtained or derived from any suitable source. As noted above, that source may be natural or synthetic. In certain embodiments, the polyunsaturated fatty acid-containing oil can include fish oil. In other embodiments, the polyunsaturated fatty acid-containing oil can be, for example, from an algal source, from a marine organism, or a fermentation product of a microorganism.

Suitable waxes can be of natural or synthetic origin. In some embodiments, the wax includes a wax ester. Generally, there are two types of waxes: hydrocarbon waxes and wax esters. Hydrocarbon waxes are typically petroleum-derived long hydrocarbon chains without any functional group, although some hydrocarbon waxes have natural biological sources. Wax esters are typically plant-derived or animal-derived, and are esters of long fatty acids and long fatty alcohols.

Suitable wax esters can include a major fatty acid chain length of at least 14 carbons to no more than 36 carbons. Thus, in certain embodiments, a suitable wax ester can include a major fatty acid chain length of at least 16 carbons, at least 22 carbons, at least 24 carbons, or at least 26 carbons. Also, in certain embodiments, suitable wax esters can include a major fatty acid chain length of no more than 32 carbons, no more than 26 carbons, or no more than 24 carbons.

Suitable wax esters can include a major fatty alcohol length of at least 20 carbons and no more than 40 carbons. Thus, in certain embodiments, a suitable wax ester can include a major fatty alcohol length of at least 24 carbons, at least 28 carbons, at least 30 carbons, or at least 32 carbons. Also, in certain embodiments, a suitable wax ester can include a major fatty acid alcohol length of no more than 38 carbons, of no more than 34 carbons, no more than 32 carbons, or no more than 30 carbons.

Suitable wax esters can have a melting point of at least 60° C. and no more than 90° C. Thus, in certain embodiments, a suitable wax ester can have a melting point of, for example, at least 60° C., at least 70° C., or at least 75° C. Also, in certain embodiments, a suitable wax ester can have a melting point of, for example, no more than 90° C., no more than 85° C., or no more than 83° C.

In some embodiments, the composition can include wax in an amount of at least 0.1% and no more than 15% of the composition, by weight. Thus, in certain embodiments, the composition can include wax in an amount of at least 0.1%, at least 0.2%, or at least 0.5% of the composition, by weight. Also, in certain embodiments, the composition can include wax in an amount of more than 2.5%, no more than 5%, or no more than 15%, by weight.

In certain embodiments, the wax ester can include rice bran wax. Rice bran wax is a natural wax ester derived from rice bran produced from milling rice (Oriza sativa). Most rice varieties are composed of roughly 20% rice husk, 11% bran layers, and 69% starchy endosperm. In certain rice milling processes, the husk is removed in a first step, yielding brown rice. Brown rice may be further processed to remove the bran, yielding the bran and refined grains of white rice. The primary constituents of rice bran are protein, fiber, and oil.

Rice bran contains about 20 wt % oil, which can be extracted with an organic solvent such as, for example, hexane. Along with the oil, other substances that may be extracted from rice bran include, for example, free fatty acids, partial glycerides, phospholipids, and other unsaponifiable materials, such as, for example, certain antioxidants (e.g., tocopherol, oryzanol, and tocotrienol), squalene, and wax esters. Rice bran oil contains a plurality of components such as, for example, rice bran wax, antioxidants, and sterols. The concentration of rice bran wax in the oil typically ranges from 1% to 3%.

Rice bran wax is a high melting solid that settles from the rice bran oil upon cooling. Rice bran wax is separated by filtration and refined by bleaching and deodorization. Refined rice bran wax also may contain amounts of free fatty acids and/or antioxidants such as, for example, oryzanol and tocopherols. The primary components of rice bran wax are long chain fatty acids esterified to very long chain fatty alcohols. (Vali et al., A process for the preparation of food grade rice bran wax and the determination of its composition, JAOCS, 82, 57-64, 2005). The composition of typical rice bran wax esters includes a fatty acid shown in Table 1, below, esterified to a fatty alcohol shown in Table 1.

TABLE 1

| Carbon Number | Fatty Acid Wt. % | Fatty Alcohol Wt. % |
|---|---|---|
| 16 | 3.6 | — |
| 18 | 2.3 | — |
| 20 | 5.3 | — |
| 22 | 26.1 | — |
| 24 | 40.5 | 3.5 |
| 26 | 11.5 | 8.4 |
| 28 | 6.6 | 14.0 |
| 30 | 3.1 | 26.4 |
| 32 | 1.0 | 19.6 |
| 34 | — | 16.5 |
| 36 | — | 9.2 |
| 38 | — | 2.4 |

A typical rice bran wax ester structure can include any of the fatty acids shown in Table 1 (e.g., C16-C32) esterified to any of the fatty alcohols shown in Table 1 (e.g., C24-C38). Thus, a for example, typical rice bran wax ester structure can include a total carbon chain length of about 40 carbons or more (e.g., a C16 fatty acid ester of a C24 fatty alcohol) up to and including about 70 carbons (e.g., a C32 fatty acid ester of a C38 fatty alcohol). In one particular embodiment, a typical rice bran wax ester structure includes lignoceric acid (C24 fatty acid) ester of tricontanol (C30 fatty alcohol)—i.e., a total carbon chain length of 54 carbon atoms, having a molecular formula of C₅₄H₁₁₈O₂ and a molecular weight of 798.

Rice bran wax is natural hard wax with high melt point. Rice bran wax contains long chain fatty alcohols esterified to fatty acids. Long hydrocarbon chains of fatty acids and fatty alcohols provide hydrophobic character, while the polar ester functionality provides hydrophilic character. The molecular structure of rice bran wax facilitates packing in the solid state to form a hard high melting wax. When the wax ester is dissolved in hydrophobic liquids and cooled, the wax molecules readily solidify into a thin crystalline mesh that can entrap a large volume of liquid. Such compositions are referred to as organogels.

Rice bran wax can be useful for changing the rheology of a formulation when incorporated at relatively low concentration compared with other natural waxes. Rice bran wax-containing formulations can exhibit thixotropy with enhanced viscosity, help stabilize emulsions, and improve shelf-stability of the formulation. Rice bran wax can act as a moisture barrier, and can add gloss and/or luster to surfaces to which it is applied, which can be desirable in many food and cosmetic applications. In manufacturing, rice bran wax can provide friction modification and gloss enhancing properties, which can be useful in certain pharmaceutical applications such as, for example, tablet panning and coating. Also, plasticizing and mold release properties of rice bran wax can make it useful in polymer compounding formulations.

Carnauba wax (CX) is derived from leaves of carnauba palm (Copernicia prunifera), native to northeastern Brazil. Carnauba wax contains mainly esters of fatty acids and fatty alcohols and minor amounts of hydrocarbons, hydroxy fatty acids, and cinnamic acid derivatives. It has a melting point of 80° C., which is comparable to rice bran wax. Both carnauba and rice bran wax are high melting hard waxes and used in certain similar applications.

In certain embodiments, the wax ester can be extracted from a natural source and, therefore, may contain certain amounts of antioxidants such as, for example, tocopherol, tocotrienol, oryzanol, or any combination thereof. Thus, the composition can include at least one antioxidant. As just noted, the antioxidant can be a component of the wax ester. In other embodiments, one or more antioxidants can be added to the composition, whether or not any natural antioxidant is present in the wax ester extract. An added antioxidant can be naturally-occurring, such as, for example, a tocopherol, a tocotrienol, green tea extract, rosemary extract, sesame extract, oryzanol, or oryzanol-containing rice bran oil. An added antioxidant can also be a synthetic antioxidant such as, for example, butylated hydroxyanisole (BHA), butylated hydroxy toluene (BHT), and tert-butylhydroquinone (TBHQ). Also, any combination of antioxidants can be used, including any combination of natural antioxidants, any combination of synthetic antioxidants, or any combination of natural and synthetic antioxidants.

The composition may be effective for limiting the extent to which the polyunsaturated fatty acid-containing oil in the composition is oxidized. The oxidation limiting property of the composition may be demonstrated in any suitable manner. One suitable way of demonstrating limited oxidation of the polyunsaturated fatty acid-containing oil is to measure the formation of peroxide over time and compare against a control composition of the same oil without antioxidant or wax in the composition. Suitable compositions can decrease oxidation of the polyunsaturated fatty acid-containing oil so that peroxide formation is, for example, 90% or less of that observed in a control composition after 28 days at 45° C. In certain embodiments, the composition can decrease oxidation of the polyunsaturated fatty acid-containing oil so that peroxide formation is, for example, no more than 82%, no more than 30%, no more than 22%, or no more than 18% of that observed in a control after 28 days at 45° C.

Another way of determining whether the composition limits oxidation of polyunsaturated fatty acid-containing oil is to assess the organoleptic properties of the composition over time. Suitable compositions can reduce undesirable organoleptic properties and/or delay the onset of undesirable organoleptic properties such as, for example, taste or odor.

The composition may adopt a crystalline structure, a gel structure, or form an emulsion. Additionally, the wax may encapsulate the oil. For example, Table 4 shows the gelation properties of rice bran wax. Rice bran wax forms gel structure in liquid oils at concentrations as low as 0.5%. The rice bran wax gel formed in Example 2 can encapsulate liquid oil. Table 5 shows that rice bran wax possesses high gel-flow temperatures at low concentrations. These rice bran wax gels possess high gel strength, a desirable characteristic for encapsulation. Microencapsulated oils prepared in this way can be combined with one or more ingestible carriers such as, for example, carbohydrates including but not limited to modified cellulose and maltodextrins. Such preparations in which a liquid omega-3 fatty acid-containing oil is encapsulated in a wax can reduce the undesirable organopletic properties associated with certain omega-3 fatty acid-containing oils (e.g., the “fishy” aftertaste or odor associated with certain oils).

If the composition is composed of materials that are safe for ingestion, the composition may be a component (e.g., an additive or coating) of a nutritional supplement, food, or cosmetic product. Food products may include solid, semisolid, gelatinous, or liquid products, and may be frozen or unfrozen. Thus, suitable foods can include, for example, beverages, dairy, breakfast cereals, snack bars, energy bars, drink mixes, dough, breads, pizza, snacks, biscuits, other food ingredients, and the like.

Generally, the composition may be formed by mixing a polyunsaturated fatty acid-containing oil and melted wax; and allowing the mixture to cool. In some embodiments, the wax may be melted prior to mixing with the oil. In other embodiments, the wax and the oil may be combined prior to heating.

In certain embodiments, the method can further include deoxygenating the melted wax/oil mixture. In other embodiments, the method can further include homogenizing the melted wax/oil mixture.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Materials and Methods:

Fish oil is refined menhaden oil obtained from Omega Protein Inc (Reedville, Va.). Unless otherwise indicated, the menhaden oil contains no added antioxidants. Where indicated, the menhaden oil contained 500 ppm of mixed tocopherols and 200 ppm of tert-butylhydroquinone (TBHQ). The typical composition of refined menhaden oil is provided in Table 2.

TABLE 2

| Material/Specification | Typical value |
|---|---|
| Cholesterol (%) | 0.2-0.5 |
| Color (Grdner) | 2-3 |
| Free fatty acids (%) | 0.06-0.1 |
| Anisidine number | 6-8 |
| Peroxide value (meq/kg) | 1-3 (maximum 3) |
| EPA, C20:5 (%) | 10 |
| DHA, C22:6 (%) | 10 |
| Total Omega 3 polyunsaturated fattyacids (%) | 30 |

Olive oil is refined edible oil obtained from a grocery store. Rice bran wax is obtained from Global Agritech, Inc. (Minneapolis, Minn.). The typical properties of rice bran wax used in the following Examples are shown in Table 3. The typical analysis given in the table is a snap shot of analysis results of a sample and the expected variation of each property is shown as range.

TABLE 3

| Property | Typical Analysis | Range |
|---|---|---|
| Melt Point (° C.) | 80 | 78-81 |
| Acid Value (mg KOH/g) | 3.7 | 2-6 |
| Iodine Value | 2.1 | 2-8 |
| Saponification Value (mg KOH/g) | 85.3 | 78-88 |
| Penetration Depth (25° C., dmm) | 2 | 2-4 |
| Color (Gardner Scale) | 5 | 4-6 |

Carnauba wax is supplied by Lambent Technologies (Gurnee, Ill.).

Sucrose oligoesters were obtained from Mitsubishi Food Corporation (Tokyo, Japan).

Polyglycerol fatty acid esters were supplied by Sakamoto Pharmaceuticals (Osaka, Japan).

An Olympus BX-50 equipped with an Olympus PM-20 camera was used for microscopic observations. The crystal morphology observations were made with cross polarized light. The x-ray diffraction pattern was measured using a Rigaku X-ray diffractometer (40 kv, 10 mA) with Cu-Kα radiation.

Peroxide value determinations were made according to the American Oil Chemists Society method, AOCS Cd 8-53. All the peroxide measurements were made on duplicate samples. The peroxide values are expressed as the average of the two measurements.

### Example 1

Crystallization and gelatin behavior of rice bran wax (RBX) and carnauba wax (CX) in liquid oil (olive oil) at different concentrations were tested. Crystallization times were determined by preparing the wax mixtures in oil, heating the mixture, and allowing the mixture to cool. The oil/wax mixtures were prepared by the addition of appropriate amounts of solid wax to 15 grams of olive oil in 2.5 cm diameter test tubes. Each oil/wax mixture was heated to 80° C. in a water bath with shaking to dissolve the wax in the liquid oil. The resulting homogeneous solutions were left at room temperature (at 20° C.) to cool. The olive oil/wax solutions were observed without disturbing until the appearance of crystals. The times taken for various concentrations of rice bran wax and carnauba wax for first crystal appearance are shown in Table 4.

TABLE 4

| Wax Concentration (%) | 4 | 2 | 1 | 0.5 |
|---|---|---|---|---|
| RBX (minutes:seconds) | 0:34 | 1:03 | 1:38 | 2:40 |
| CX (minutes:seconds) | 0:35 | 1:03 | 1:26 | 2:05 |

Thus, the crystallization of rice bran wax in liquid oil occurs within few minutes.

### Example 2

The gelation times of oil/wax mixtures were determined in a similar manner. The oil/wax mixtures were prepared as described in Example 1. The homogenous oil/wax solutions were left at 20° C. for gelation. The gelation time in minutes were measured as the time necessary for the oil to stop flowing, upon tilting the sample to 45°. The results are shown in Table 5.

TABLE 5

| Wax Concentration (%) | 4 | 2 | 1 | 0.5 |
|---|---|---|---|---|
| RBX (minutes:seconds) | 4:42 | 6:24 | 7:16 | 10:45 |
| CX (minutes:seconds) | 13:45 | * | * | * |

* No gelation was observed in two days.

### Example 3

Oil/wax mixtures were prepared by adding the appropriate amounts of solid wax to 15 grams of olive oil. The oil wax mixture is heated to 80° C. in a water bath with agitation to dissolve the wax in the liquid. The resulting homogeneous solutions are left at room temperature (at 20° C.) to cool and form gel. The gels were left at room temperature for two to three hours. The gelled samples were heated in a water bath from 20° C. to 70° C. at 3° C./minute. The temperature at which flow is observed upon tilting the sample to 45° is taken as gel-flow temperature. The results are shown in Table 6.

TABLE 6

| Wax Concentration (%) | 4 | 2 | 1 |
|---|---|---|---|
| RBX (° C.) | 64.8 | 59.8 | 57.9 |
| CX (° C.) | 59.1 | * | * |

* No gelation.

### Example 4

A 1% rice bran wax/olive oil mixture and a 1% carnauba wax/olive oil mixture were prepared by adding an appropriate amount of wax to olive oil and heating to 80° C. to form a homogenous solution. The solutions were cooled to room temperature until wax crystals formed. A small sample containing crystals were placed on a slide and covered with a cover slip for microscopic observation.

The microscopic observations were made under cross polarized light and revealed the morphology of rice bran wax crystals as very long needles of 20 micrometers (μm) to 50 μm, a desirable feature for gel formation. The carnauba wax crystals were spherulitic having a diameter of less than 10 μm.

Neat powder x-ray diffraction of rice bran wax revealed a β′ subcell structure with very strong wide angle short spacings of 4.14 and 3.74 Å, characteristic of orthorhombic subcell packing of this polymorphic form. The x-ray diffraction long spacings of the powder indicated a weak diffraction corresponding to 70 Å. The long spacing roughly corresponds to the typical wax ester structure of lignoceric acid (C24) esterified to C30 alcohol, tricontanol. The straight 52 carbon chain distance corresponds to 66 Å and the ester function with two carbons occupies about 4 to 5 Å. The gels made from 4% rice bran wax in olive oil revealed similar short spacings at 4.17 and 3.75 Å indicating the rice bran wax gels have an orthorhombic β′-sub cell structure. It is known that the long chain fatty acid esters in the solid state pack in three different sub cell structures corresponding to α-, β′-, and β-polymorphs with increasing melting temperatures. (Kodali et al, Molecular packing in triacyl-sn-glycerols: Influence of acyl chain length and unsaturation. J. Dispersion Sci. Technol. 10, 393-440, 1989). Of the three sub cells the β′-polymorph crystal structure offers better properties to the food functionality.

### Example 5

Emulsions were prepared with and without rice bran wax as follows. Emulsifiers sucrose oligoesters (0.5 g) and polyglycerol fatty acid ester (0.5 g) were mixed with olive oil (100 g) by heating to about 80° C. Distilled water (25 ml) was added to the oil/emulsifier mixture and mixed at 4000 rpm with a high speed mixer for 10 minutes. The emulsion containing rice bran wax was prepared similarly except that 1.5 g of wax was added to the oil/emulsifier mixture. The oil/emulsifiers/wax mixture was heated to melt the wax and homogenized before subjecting it to a high speed mixer at 4000 rpm. The emulsions thus prepared were aliquoted into 25 ml vials for observation. The emulsions containing rice bran wax remained homogenous without phase separation for three months. The emulsions without the rice bran wax started phase separating in about a day.

### Example 6

Refined menhaden oil (100) grams was deoxygenated to remove the dissolved oxygen by applying vacuum and flushed with nitrogen. The deaeration and inert gas flushing were repeated three times. The oil was heated, alone (Control), with 1.5 g rice bran wax (RBX-1.5), or with 1.5 g carnauba wax (CX-1.5) to about 80° C. with agitation and divided into five vials before being cooled to room temperature. The samples were incubated at 45° C. with a loose cap. The peroxide value of one vial of each mixture (oil alone, oil/rice bran wax, and oil/carnauba wax) was measured immediately and at 7-day intervals for a period of four weeks. Results are shown in Table 7.

TABLE 7

| | | | | Peroxide Value | | | | |
|---|---|---|---|---|---|---|---|---|
| | Fish oil | RBX | CX | 0 | 7 | 14 | 21 | 28 |
| Example | (g) | (g) | (g) | days | days | days | days | days |
| Control | 100 | — | — | 1.26 | 21.0 | 36.0 | 55.0 | 54.1 |
| RBX-1.5 | 100 | 1.5 | — | 1.53 | 10.5 | 11.0 | 15.3 | 15.3 |
| CX-1.5 | 100 | — | 1.5 | 0.88 | 15.4 | 32.8 | 46.9 | 43.9 |

### Example 7

Refined menhaden oil (100 g) containing antioxidants (500 ppm of mixed tocopherols and 200 ppm of TBHQ) was deoxygenated to remove the dissolved oxygen by applying vacuum and flushed with nitrogen. The daeration and inert gas flushing was repeated three times. The oil was heated, alone (RBX-0), with 0.5 g rice bran wax (RBX-0.5), or with 1.5 g rice bran wax (RBX-1.5) to about 80° C. with agitation and divided into five vials before being cooled to room temperature. The samples were incubated at 45° C. with a loose cap. The peroxide value of one vial of each mixture (oil alone, oil/rice bran wax) was measured immediately and at 7-day intervals for a period of four weeks. The results are shown in Table 8 with the Control (oil without antioxidants, no wax added) from Table 7 for comparison.

TABLE 8

| | | | Peroxide Value | | | | |
|---|---|---|---|---|---|---|---|
| | Fish oil | RBX | 0 | 7 | 14 | 21 | 28 |
| Example | (g) | (g) | days | days | days | days | days |
| Control | 100 | — | 1.26 | 2.10 | 36.0 | 55.0 | 54.1 |
| RBX-0 | 100* | — | 0.26 | 7.53 | 21.9 | 11.8 | 14.2 |
| RBX-0.5 | 100* | 0.5 | 0.27 | 3.93 | 4.30 | 8.47 | 9.78 |
| RBX-1.5 | 100* | 1.5 | 0.57 | 3.31 | 3.48 | 6.09 | 11.9 |

*Contains added antioxidants, 500 ppm of mixed tocopherols and 200 ppm of TBHQ.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of stabilizing polyunsaturated fatty acid-containing oils, the method comprising:
mixing a polyunsaturated fatty acid-containing oil and melted wax; and
allowing the mixture to cool.

2. The method of claim 1 wherein the oil comprises an omega-3 fatty acid-containing oil.

3-4. (canceled)

5. The method of claim 1 wherein the wax comprises a wax ester.

6. The method of claim 1 wherein the wax comprises an antioxidant.

7. The method of claim 6 wherein the antioxidant comprises a natural antioxidant.

8. (canceled)

9. The method of claim 1 wherein the wax comprises a major fatty acid chain length of at least 14 carbons to no more than 36 carbons and the wax comprises a major fatty alcohol length of at least 20 carbons and no more than 40 carbons.

10-12. (canceled)

13. The method of claim 1 wherein the wax comprises rice bran wax.

14. (canceled)

15. The method of claim 1 wherein mixing a polyunsaturated fatty acid-containing oil and melted wax:
combining the wax and the oil;
heating the combination to melt the wax; and
homogenizing the mixture.

16. A composition comprising:
a oxidizable polyunsaturated fatty acid-containing oil; and
a wax capable of reducing oxidation of the oil.

17. The composition of claim 16 wherein the oil and the wax are
ingestible and the oil is encapsulated in the wax.

18. The composition of claim 16 wherein the oil comprises an omega-3 fatty acid-containing oil.

19-21. (canceled)

22. The composition of claim 16 wherein the wax comprises a wax ester.

23. The composition of claim 16 wherein the wax comprises an antioxidant.

24. The composition of claim 23 wherein the antioxidant comprises a natural antioxidant.

25. (canceled)

26. The composition of claim 16 wherein the wax comprises a major fatty acid chain length of at least 14 carbons to no more than 36 carbons and a major fatty alcohol length of at least 20 carbons and no more than 40 carbons.

27-28. (canceled)

29. The composition of claim 16 wherein the wax comprises rice bran wax.

30-33. (canceled)

34. The composition of claim 16 wherein the wax comprises at least 0.1% and no more than 15% of the composition, by weight.

35. The composition of claim 16 wherein the composition is an emulsion.

36. The composition of claim 35 wherein the polyunsaturated fatty acid-containing oil comprises an omega-3 fatty acid-containing oil, the wax comprises a wax ester, and further comprising at least one emulsifier.

37-38. (canceled)

39. A method of preparing an encapsulated liquid oil, the method comprising:
preparing a mixture comprising:
a liquid oil, and
a melted wax at a concentration effective to provide a gel-flow temperature of at least 50° C.; and
allowing the mixture to cool until at least a portion of the wax encapsulates at least a portion of the oil.

40-51. (canceled)
